(19) United States
(12) Patent Application Publication
(10) Pub. No.: US 2003/0216622 A1
(43) Pub. Date: Nov. 20, 2003

(54) Device and method for orienting a device in vivo

(76) Inventors: Gavriel Meron, Petach Tikva (IL); Arkady Glukhovsky, Santa Clarita, CA (US); Harold Jacob, Jerusalem (IL); Shlomo Lewkowicz, Kiryat Tivon (IL)

Correspondence Address: Eitan, Pearl, Latzer & Cohen Zedek, LLP., Suite 1001, 10 Rockfeller Plaza, New York, NY, 10020, US

(21) Appl. No.: 10/423,023

(22) Filed: Apr. 25, 2003

Related U.S. Application Data
(60) Provisional application No. 60/375,006, filed on Apr. 25, 2002.

(51) Int. Cl. A61B 5/00

(52) U.S. Cl. 600/300

(57) ABSTRACT

An in vivo device, such as an in vivo imaging device or other sensing device, may include a device body and at least one appendage coupled to the device body. According to some embodiments the appendage(s) may be extended or expanded, or reduced or removed, in vivo, thereby altering the device geometry while in a body lumen.

### RELATED APPLICATION DATA

The present application claims benefit from prior provisional application serial No. 60/375,006 filed on Apr. 25, 2002 and entitled “METHOD FOR POSTIONING AN OBJECT IN A BODY LUMEN.”

### FIELD OF THE INVENTION

The present invention relates to a method for establishing the orientation of an in vivo device with respect to a body lumen and to a device capable of being oriented in vivo.

### BACKGROUND OF THE INVENTION

In vivo sensors, including image, sensors are typically non-invasive tools used in diagnosis of body systems. For example, devices (e.g., swallowable devices) may be used for sensing in vivo conditions in the gastrointestinal (GI) tract, such as, for example, temperature, pH electrical activity, impedance or pressure. Imaging devices can be used for sensing the GI tract.

A sensing device such as a capsule which includes a sensor may be swallowed and moved through the small intestine passively (e.g. by peristalsis) or actively while sensing the small intestine. A sensor may be any sensor including an image sensor. However, passive movement of a device through larger body lumens, such as, the stomach or the large intestine may be slow, tumbling and unpredictable. Furthermore, the device may become trapped in a fold of a wall of the body lumen. In such a position, an imaging device (which may include illumination) may not have a sufficiently wide field of image and/or field of illumination to obtain images suitable for diagnostic purposes. When using a capsule to sense a physiological parameter, diagnose, or treat an area in a larger body lumen, the capsule may not be oriented properly with respect to the lumen (e.g., along the lumen) so that proper sensing or treatment can be accomplished. In these cases monitoring, diagnosing, and treating larger body lumens may be not efficient.

Current methods of moving and positioning objects, especially sensing devices, in large body lumens, such as, the large intestine, usually include, for example, use of push-endoscopes and catheters. These devices, however, are inconvenient for patient use, and do not always enable reaching distal parts of the body lumen.

### SUMMARY OF THE INVENTION

It is accordingly the object of the present invention to provide an in-vivo device capable of passing through a body lumen and whose geometry can alter or be altered (for example, expanded and/or contracted). In accordance with an embodiment of the invention an in vivo device may include a device body and at least one appendage coupled to the device body. According to an embodiment of the invention the appendage is expandable (for example, may be extended or collapsed). According to some embodiments the appendage(s) may be extended in vivo, thereby altering the device geometry while in a body lumen. According to one embodiment the geometry of an in vivo device may be altered for the purpose of establishing an orientation and position of that device with respect to the body lumen through which it is passing.

According to some embodiments the device may be a sensing device, a diagnostic device, a therapeutic device, or a combination thereof. Typically the device is able to progress passively through a body lumen, such as through the entire GI tract. Alternatively, the device may be propelled or otherwise guided through any body lumen.

### BRIEF DESCRIPTION OF THE DRAWINGS

The invention, both as to organization and method of operation, may best be understood by reference to the following detailed description when read with the accompanied drawings in which:

FIG. 1A schematically illustrates a longitudinal cross sectional view of an in vivo device with appendages according to an embodiment of the invention;

FIG. 1B depicts an image receiving and display system according to one embodiment of the invention;

FIG. 2 schematically illustrates an in vivo device with a ring or torroid shaped appendage positioned around the device according to an embodiment of the invention;

FIG. 3 schematically illustrates an in vivo device with a set of appendages according to an embodiment of the invention;

FIG. 4A schematically illustrates an in vivo device with a plurality of asymmetrical appendages according to an embodiment of the invention;

FIG. 4B schematically illustrates an in vivo device with a ring or torroid shaped appendage surrounding the device asymmetrically according to an embodiment of the invention;

FIG. 5 schematically illustrates an in vivo device oriented at an angle with respect to a body lumen, according to an embodiment of the invention;

FIGS. 6A and 6B are cross sectional views of an in-vivo device with an expandable appendage, according to one embodiment of the invention;

FIGS. 7A and 7B are cross sectional views of a device with an expandable appendage, according to one embodiment of the invention;

FIGS. 8A and 8B are schematic longitudinal cross sectional views of a device with an appendage that substantially surrounds or encompasses the device body, according to one embodiment of the invention;

FIGS. 9A and 9B depict an in-vivo device surrounded by a layer of material, and with the material removed, according to one embodiment of the invention; and

FIG. 10 is a flow chair depicting the steps of a method according to one embodiment of the invention.

### DETAILED DESCRIPTION OF THE INVENTION

In the following detailed description, numerous specific details are set forth in order to provide a thorough understanding of the invention. However, it will be understood by those of ordinary skill in the art that the present invention may be practiced without these specific details. In other instances, well-known methods, procedures, components and circuits have not been described in detail so as not to obscure the present invention.

Some embodiments of the present invention are directed to a typically swallowable device that may passively or actively progress through the gastro-intestinal (GI) tract, pushed along by natural peristalsis. Other embodiments are directed at in vivo sensing devices that may be passed through other body lumens such as through blood vessels, the reproductive tract, etc. The device may be a sensing device, a diagnostic device, a therapeutic device, or a combination thereof. According to one embodiment the device may include an image sensor. Devices according to embodiments of the present invention may be similar to embodiments described in International Application WO 01/65995 and/or in U.S. Pat. No. 5,604,531, each of which are assigned to the common assignee of the present invention and each of which are hereby incorporated by reference. Of course, devices as described herein may have other configurations and sets of components.

According to some embodiments of the present invention, the device may be configured to change its shape or geometry when entering certain parts of the GI tract, for example voluminous lumens, such as the stomach and/or large intestine, so that it may be better adjusted to movement and sensing through a voluminous body lumen.

According to an embodiment of the invention symmetrically increasing the volume around a device may help center the device along the lumen and increase its coverage of the intestinal surface. When progressing in a large body lumen such as the large intestine or the stomach, the device may be positioned substantially in the middle of the lumen, oriented along the lumen and away from the walls, thus enabling facile flow of the device along the GI tract. Furthermore, being in this position, the illumination field provided by one or more illumination sources may be large enough to enable efficient imaging even distant objects or features. Also, in this position, the imaging device may provide a wide field of view and large overlapping area between the field of illumination and the field of view, thus enabling images of the walls to be obtained without optical obstructions.

According to yet another embodiment asymmetrically increasing the volume of the device may help position the device, for example, at a specified distance away from the wall of a body lumen, straight up against the wall, or at angle with respect to the wall for the purpose of, for example, sensing, performing diagnoses, administrating medication, etc.

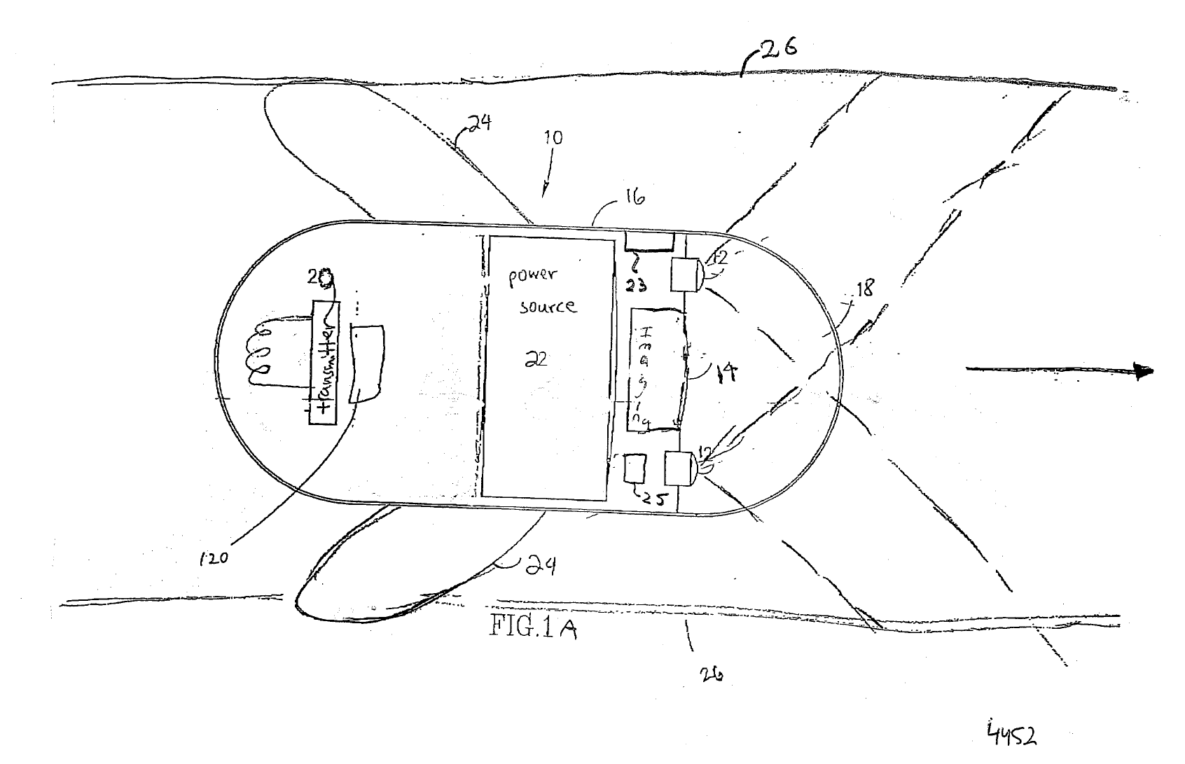

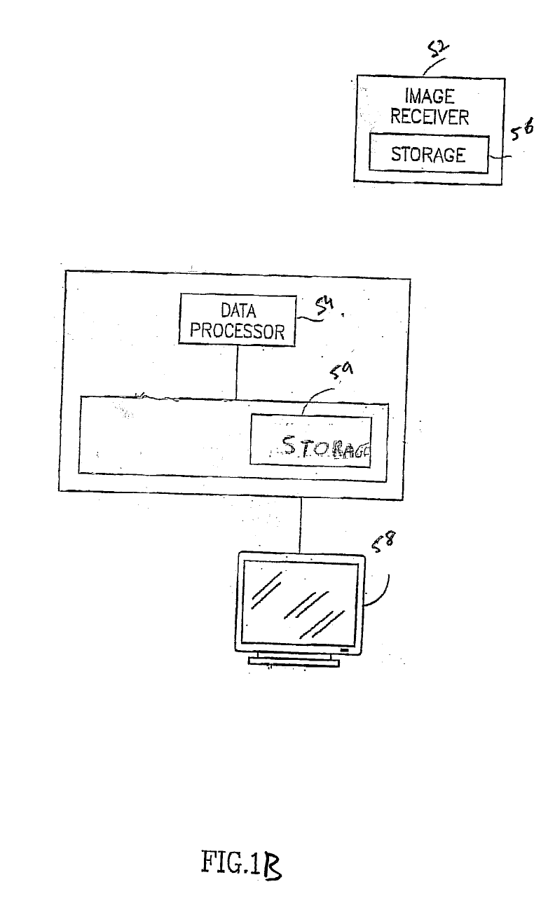

Reference is now made to FIG. 1A, which is a schematic longitudinal cross sectional view of a sensing device according to some embodiments of the invention. The sensing device 10 is a swallowable capsule shaped imaging device, but need not be swallowable, and may be other shapes. Further, sensing other than imaging may be performed. In addition, device 10 may perform other functions such as delivering medication. A device 10 may include one or more illumination sources 12 and an imaging system 14 to image body lumens, such as, the gastrointestinal tract, a transmitter 20, which may transmit image signals to an external receiving system and a power source 22, such as, a battery (e.g., silver oxide batteries, lithium batteries, or other electrochemical cells having a high energy density, or the like; other suitable power sources may be used, including sources capable of receiving energy transmitted from outside the body). Transmitter 20 may include receiver capability. A secondary sensing component 23 such as a temperature sensor, a pressure sensor, an enzymatic or other chemical sensor, an optical sensor, etc., may be included. The various components are typically enclosed within a body or housing 16. Housing 16 may typically be substantially rigid (wherein substantially rigid may include rigid). Secondary sensing component 23 is shown in one position and having one configuration, but may be in other suitable positions and configurations, depending on the mode being sensed. Sensors other than an imaging system or sensor may be used.

Device 10 may include a location tracking device 25, such as, for example, two or more transmitting antennas, each with a different wavelength, a set of magnetic coils, etc. Location detection may also be performed based on signals from transmitter 20. Location detection need not be used.

Device 10 includes one or more (in the example shown, two, but other numbers may be used) appendages 24 attached to housing 16. Typically, the appendage(s) 24 are coupled to and extend from the housing 16. In further embodiments, the size or configuration of the appendage(s) 24 is changeable, as described below. In FIG. 1A, appendages 24 are wing-like or fin-like, but may have other suitable configurations in other embodiments.

In one embodiment, the imaging system 14 includes an imager (not shown), which may be, for example, a complementary metal oxide semiconductor (CMOS) image sensor. The CMOS imager is may be an ultra low power imager and is provided in chip scale packaging (CSP). One suitable CMOS camera may be, for example, a “camera on a chip” CMOS imager specified by Given Imaging Ltd. of Israel and designed by Photobit Corp. of California, USA, with integrated active pixel and post processing circuitry. Other types of CMOS imagers may be used. In another embodiment, another imager may be used, such as a CCD imager, or any other imager. The imager may be rectangular in shape and have the same resolution in both dimensions (e.g., a 256×256 CMOS array), but other shapes, sizes and resolutions may be used. Other sensors, sensing in other modalities (e.g., pH, pressure, etc.) may be used.

The transmitter 20, which may include components, such as, for example, a compression module, for compressing data, is typically an ultra low power radio frequency (RF) transmitter with high bandwidth input, possibly provided in chip scale packaging. The transmitter 20 may transmit via an antenna. The transmitter 20 may also include circuitry and functionality for controlling the device 10. Such control functionality may be, for example, receiving sensing information (e.g., pressure, enzymatic activity, temperature, optical detection or image analysis), and, from such sensing information, determining whether or not to extend or deploy appendages (described below), as variously described below. A separate control and/or processing unit may be used. In one embodiment, data from a secondary sensing component 23 is input to the transmitter 20 or other control device, and the transmitter 20 or other control device determines if a change in appendage deployment is required (e.g., extending or deploying appendages, or, as described further below, detaching, shrinking, dissolving, etc., the appendages).

Various environmental triggers (e.g., pH, temperature, image data) may factor into a decision to change the appendage deployment or configuration, or may cause a change. Different modes of control operation may be used. For example, the detection of a change, or a pattern of change, or a set of changes across time, may affect a change in appendage deployment. For example, a detection of a pH change between a neutral level (e.g., pH 7-8) in the esophagus and an acidic level (e.g., pH 2-3) in the stomach, or between levels in the stomach (e.g., pH 2-3) and the small intestine (e.g., pH 7-9), may result in appendages being extended or expanded. The method may include control based on the elapsed time. For example, a certain amount of time after images indicate the device is ingested may elapse before an appendage is extended, or before an appendage extension method is started.

Such calculations may be performed by a processor internal to the device (e.g., transmitter 20) or by a processor external to the device, via commands received by a receiver within the device. Combinations of different parameters may be utilized in the control algorithms.

Image analysis techniques may be used to decide when to alter the configuration of appendages. For example, images may be analyzed to determine when a device is ingested, if a device is not mobile, if a device enters a certain lumen (e.g., enters the small intestine). Image processing may detect, for example, illumination, which may indicate whether the capsule is located in a small or large organ, or inside or outside the body.

In an alternate embodiment, external sensing which may lead to a decision to extend or deploy appendages may come from imaging system 14. In a further embodiment, such control or decision may come from an external source, and in such a case transmitter 20 (or other components) may include receiver capability.

Other components and sets of components may be used in the device 10. For example, a secondary sensing component or other components need not be included. Further, the functionality of various components may be divided among other components or sets of components.

FIG. 1B depicts an image receiving and display system according to one embodiment. Typically, located outside the patient's body in one or more locations are an image receiver 52, typically including an antenna or antenna array (not shown), an image receiver storage unit 56, a data processor 54, a data processor storage unit 59, and an image monitor 58, for displaying, inter alia, images recorded by the device 10. Image receiver 52 may be, for example, a portable device worn by a patient, but may be of other configurations.

Typically, data processor 54, data processor storage unit 59 and monitor 58 are part of a personal computer or workstation, which includes standard components such as processor 54, a memory, a disk drive, and input-output devices, although alternate configurations are possible. Data processor 54 may include any standard data processor, such as a microprocessor, multiprocessor, accelerator board, or any other serial or parallel high performance data processor. Image monitor 18 is typically a conventional video display, but may, in addition, be any other device capable of providing image or other data.

In one embodiment, data from a secondary sensing component 23 is transmitted by transmitter 20 to the external receiver or processing system which in turn determines when and if to extend or deploy appendages. The external receiver or processing system may transmit control information back to transmitter 20 which in turn may transmit control information to appendages or other components.

In alternate embodiments, the data reception and storage components may be of another configuration. Embodiments of a suitable external receiving and monitoring system are described in International Application WO 01/65995 and U.S. Pat. No. 5,604,531, although monitoring and/or receiving systems having other suitable structures or functions may be used.

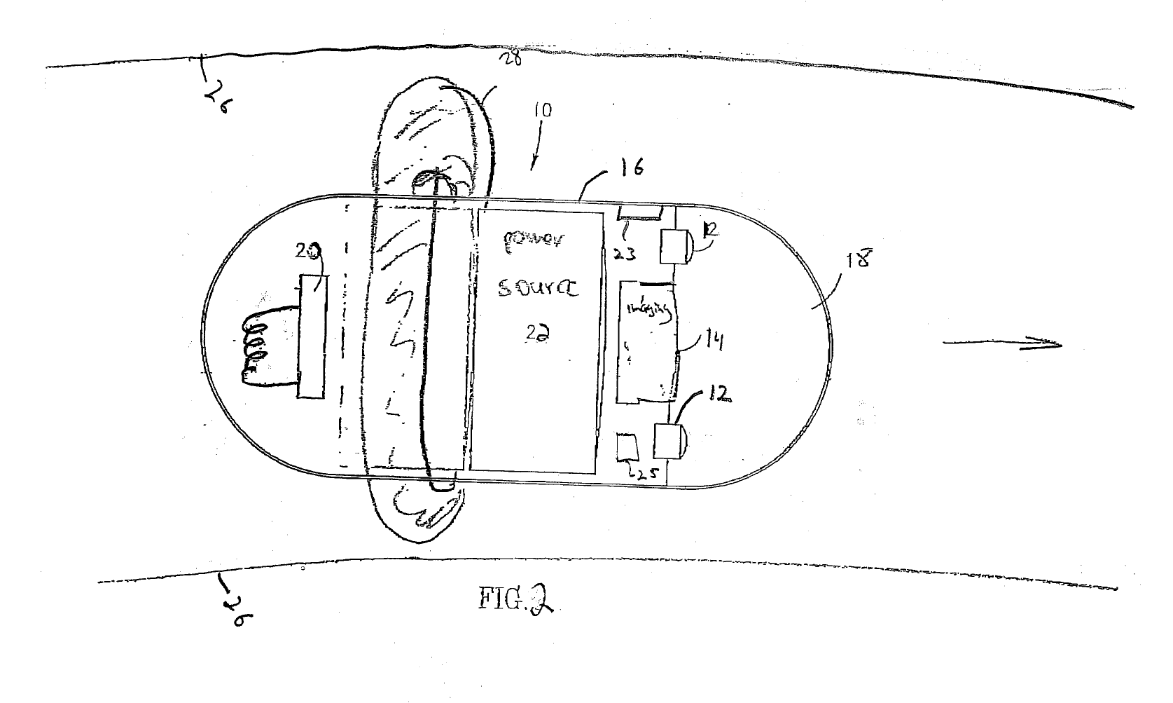

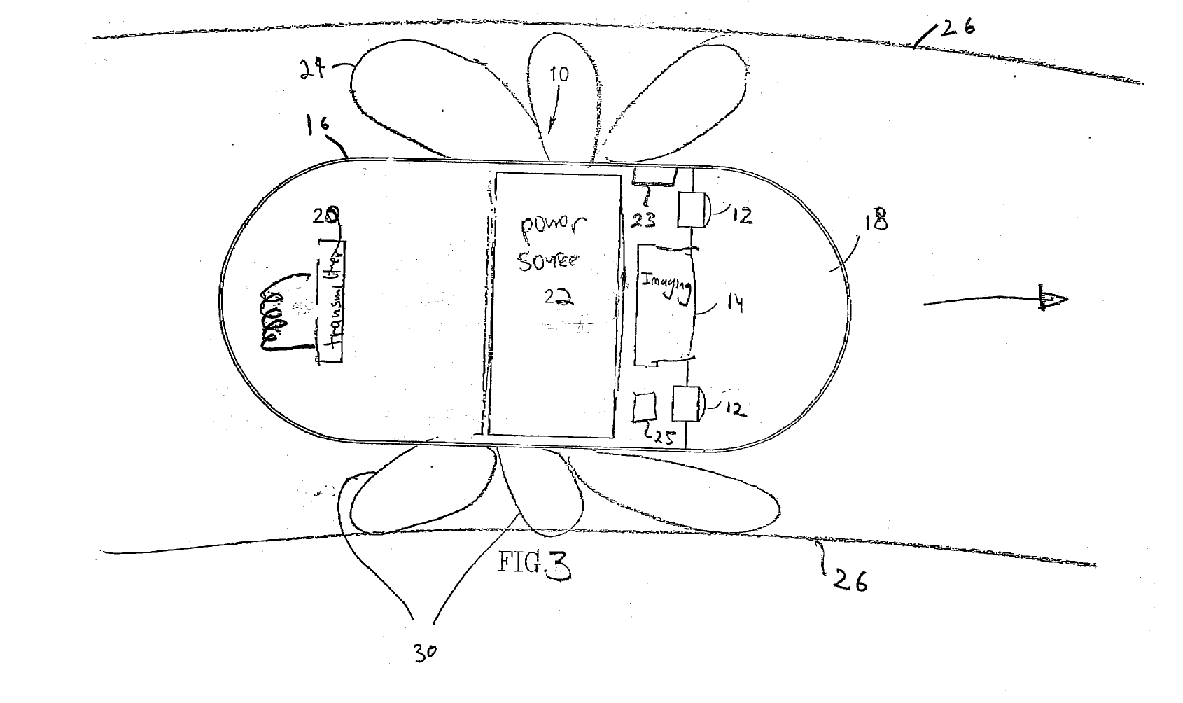

Device 10 may further include two or more wing-like or fin-like appendages 24 attached to housing 16. According to one embodiment appendages 24 may enable adjustment of the device to passive movement in large body lumens, such as the large intestine (colon) or stomach, while positioning the housing of the device 16 substantially in the center of and along the lumen. In another embodiment, a disc shaped appendage 28 surrounds the longitudinal axis of housing 16 as is shown in FIG. 2. Typically one such disc shaped appendage 28 is used, although more than one may be used in other embodiments. Alternately, multiple appendages 30 which are, for example, wing shaped, capsule shaped, cone shaped, disc shaped or a combination may be used. Various suitable shapes for appendages, which may be used in various combinations, are shown in FIG. 3. Other suitable shapes may be used.

In some embodiments, an in-vivo sensing device may be actively propelled through body lumens. In such cases, suitable appendages, such as those described herein, may also be used.

In alternate embodiments, one appendage may be used. Further, in alternate embodiments, the appendage(s) need not position the device substantially in the center of the lumen, but may position the device in other positions, for example in an off-center position.

Housing 16 may include an optical window 18 through which light or other electromagnetic radiation from illumination sources 12 may illuminate the inner portions of the body lumens. Optical window 18 may be positioned and shaped according to the shape of device 10 and according to specific imaging requirements. An optical window 18 provides a generally transparent cover for the optical elements, provides a sealed barrier to bodily fluids, and may perform other functions (such as holding optical elements). An optical system (not shown), including, for example, one or more optical elements, such as one or more lenses or composite lens assemblies, one or more suitable optical filters, or any other suitable optical elements, may aid in focusing reflected light or electromagnetic radiation onto the imager or imaging system 14 and possibly perform other light or electromagnetic radiation processing.

In one embodiment, the appendages may be situated substantially symmetrically relative to the longitudinal axis of housing 16 and their size may be such that, at certain points, the distal ends or the edges of the appendages are in close proximity to the walls 26 of the lumen. Alternatively, the ends of the appendages may slightly touch or periodically bump against the walls 26 while moving, thus keeping device 10 away from the walls. Appendages (e.g., 24, 28 or 30, or the embodiments of appendages described below) may press against the body lumen wall 26 while traversing through a lumen such as the colon, thereby dislodging or keeping the device 10 from the lumen wall. When housing 16 is situated substantially in the center and away from the lumen wall 26 of, for example, the colon, the field of illumination provided by illumination sources 12 and the field of view provided by imaging system 14 may be large enough to provide a good view of the lumen and of its walls.

In an alternate embodiment device 10 is a sensing device other than an imaging sensor, a diagnostic device, or a therapeutic device. The appendages may position housing 16 at substantially a defined position relative to the body lumen, for example, at an angle to the body lumen or parallel to the longitudinal axis of the body lumen. The housing 16 may be positioned and at a specified distance away from one of the body lumen walls. Such positioning may provide a possibly more controlled sensing, diagnosing, or treatment.

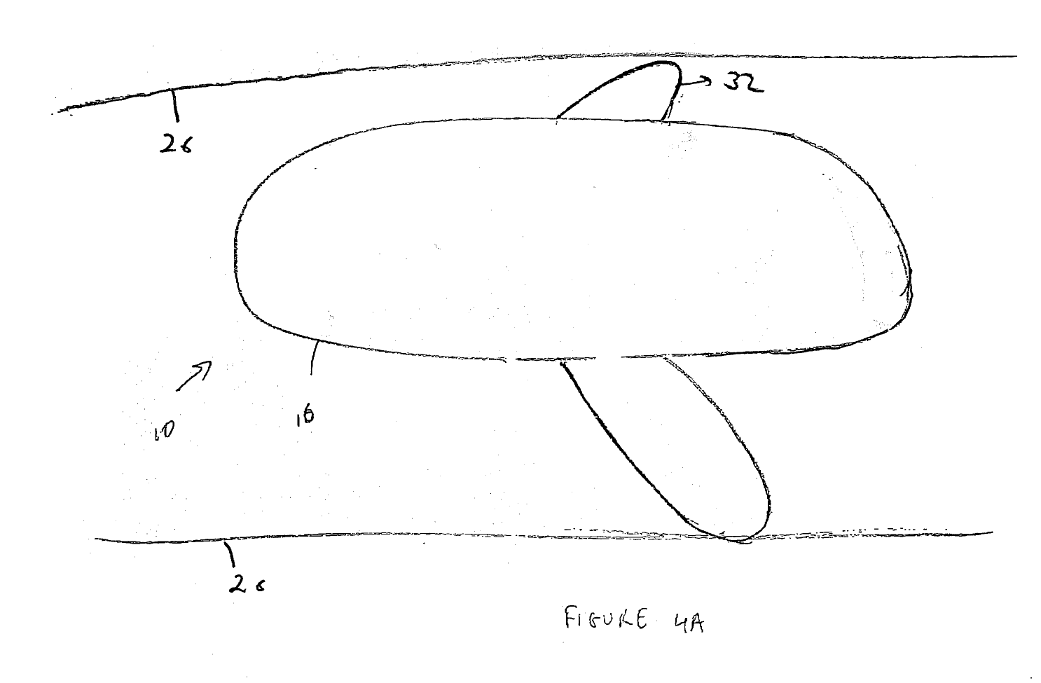

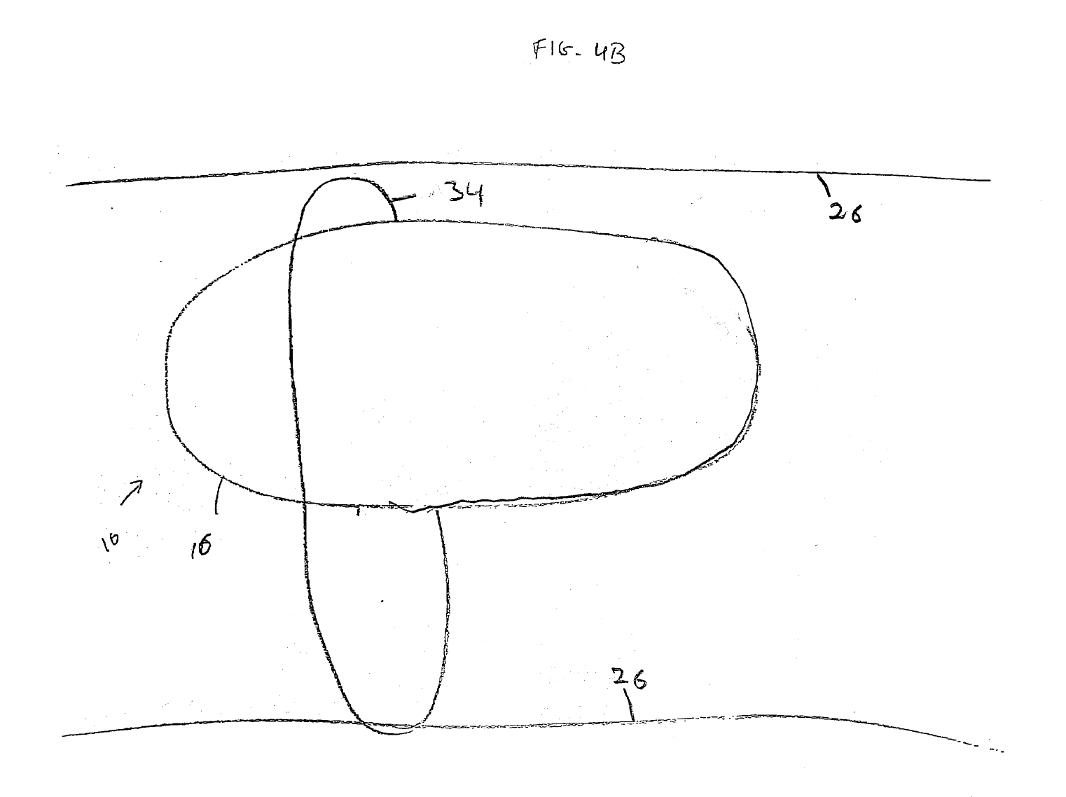

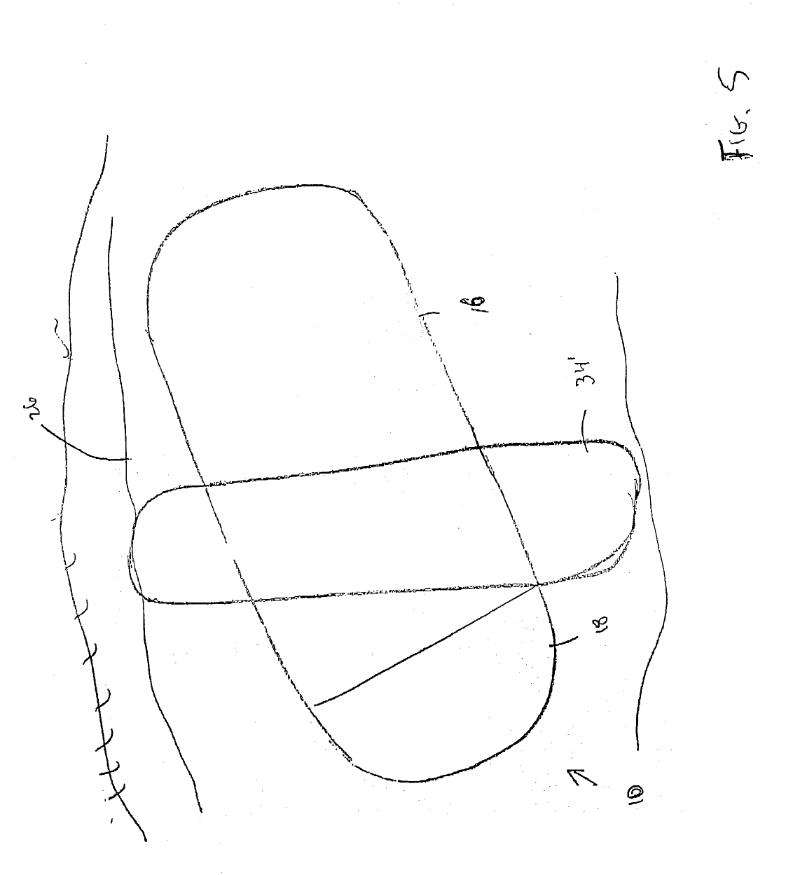

Reference is now made to FIG. 4A, FIG. 4B and FIG. 5, which are schematic longitudinal cross sectional views of a sensing device 10 with a housing 16 with asymmetrical wing shaped appendages 32, and disc shaped appendages 34 and 34′ respectively. While device 10 is typically a swallowable capsule shaped imaging device, device 10 need not be capsule shaped, and need not be swallowable. The elements of device 10, which may be similar to device 10 as described above, are not detailed so as not to obscure the figures.

The shape and size of the appendages (e.g., appendages 32, 34 and 34′, or other appendages described elsewhere herein) are typically determined such that housing 16 will be positioned at some determined distance away from a lumen wall or up against a lumen wall. As such the sensing and/or diagnosing and/or treating can be performed near or at the lumen wall. In an alternate embodiment, shown in FIG. 5, the size and shape of the appendage 34′ are determined such that the housing 16 is held at some angle with respect to the lumen wall 26. As such, the sensing portion of the device, for example the portion defined and/or behind by optical window 18, can be positioned to be pointing toward a lumen wall, for proper focusing for example.

According to one embodiment, appendages (e.g., appendages 24, 28, 30, 32, 34, 34′ and 66, described below) are made of pliant and soft material, such as, for example, rubber, hydrocarbon or silicone. The appendage may be configured in any shape that is useful for orienting and advancing the device through the body lumen through which it is traveling while not damage the walls of the body lumens. Other materials may be used, and the appendages need not be pliant or soft. Additional structures may be included in the appendages, such as hinges, springs, flexible portions, etc.

Appendages may be compactly packaged, such as rolled up or folded, in one mode, and may be extended or deployed in another mode. For example, the appendages may be released from their packaging at a desired location or time according to specific requirements. For example, device 10 may be swallowed and moved by peristalsis through the small intestine while the appendages are packaged. When device 10 enters the large intestine, the appendages may be released from their packaging and the reshaped device may be efficiently positioned or moved through the large intestine. The mechanisms by which the appendages are released from their packaging may be externally controlled. Alternatively, release of the appendages from their packaging may be automatically controlled as described below.

According to another embodiment, the appendages may be compactly packaged, such as rolled up or folded while traveling for example through the esophagus or the small intestine. The appendages may be released from their packaging, for example in the stomach by inflating the appendages or by using configurable changing material, such as bi-morph material, or by dissolving or weakening material, as described below. The inflatable appendages may be rolled and packaged to a small size. In its packaged form, device 10 may be suitable for efficiently progressing through the small intestine and in its non-packaged form; the device is most suitable for progressing through the large intestine. The inflatable appendages may contain gas-releasing granules, such as, for example, oxygen-releasing granules and crystalline sodium bicarbonate, E-2 GasII effervescent granules, commercially available from E-Z-EM Inc. of New York, USA. Typically, these granules release gas, such as, carbon dioxide or oxygen, upon contacting liquid. Appendages may contain two compartments, a first compartment containing a suitable amount of gas-releasing granules and a second compartment containing an amount of liquid, for example, 0.1 centimeter cube of water or saline. The compartments may be kept separate while device 10 is in its packaged form during its progress through the small intestine. Once the device is, for example, in the large intestine, the two compartments are merged and the drop of liquid may contact the gas-releasing granules. Gas is then released into the appendages inflating them.

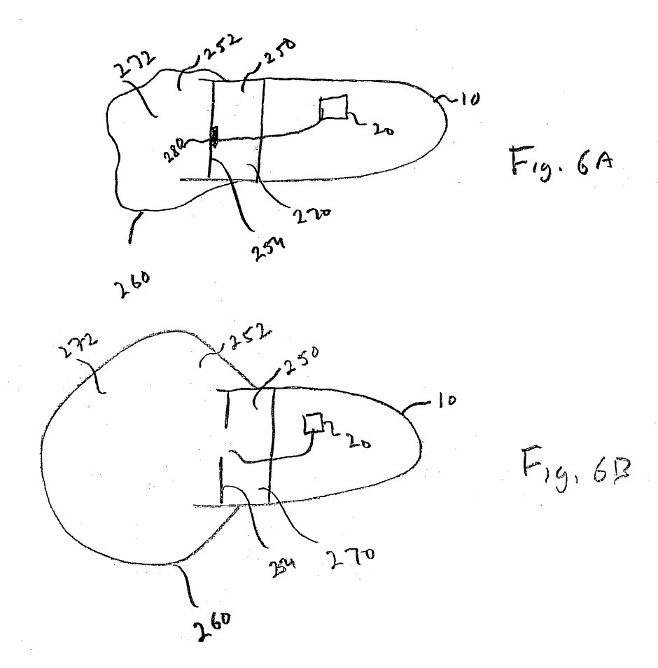

FIG. 6A is a cross sectional view of an in-vivo device with an expandable appendage. Referring to FIG. 6A, device 10 includes a controller device such as transmitter 20, and may include components similar to the embodiments of the device described elsewhere herein. Device 10 includes two chambers 250 and 252, separated by a barrier 254. One portion of the device includes a flexible and expandable covering or barrier 260 which may be constructed of, for example, rubber, plastic, corrogated or hinged material, etc. Each chamber may include a different substance 270 and 272 which, when combined, expand or produce gas (e.g., the liquid and gas producing substances described above). A signal from the control device may cause a barrier 280 between the chambers 250 and 252 to dissolve, be removed, or open (in the case the barrier 280 is a valve), allowing the materials to mix and expand or produce gas. FIG. 6B depicts an in-vivo device with an expanded appendage. In FIG. 6B the covering or barrier 260 acting as an expandible appendage has been expanded by the combination of the substances to change the shape of the device 10.

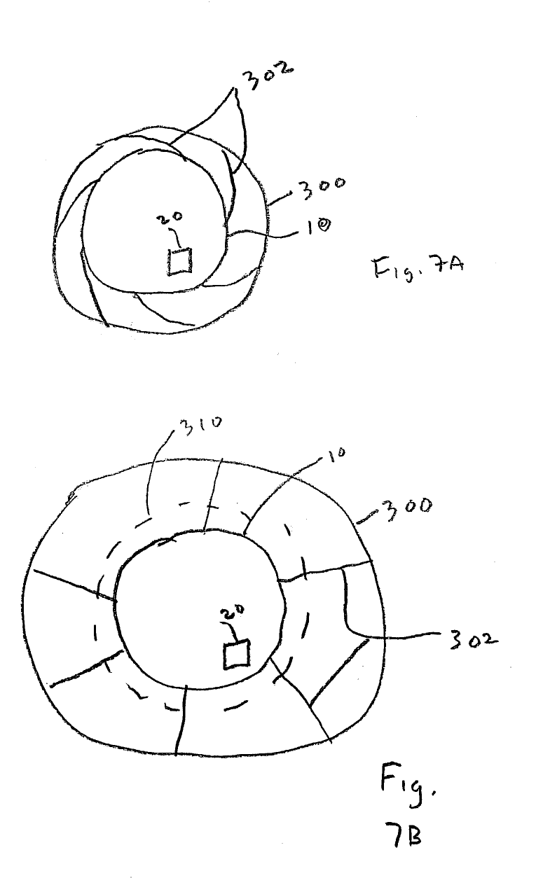

FIG. 7A is a cross sectional view of a device with an expandable appendage, according to one embodiment of the present invention. Referring to FIG. 7A, device 10 includes a controller device such as transmitter 20, and may include components similar to the embodiments of the device described elsewhere herein. Device 10 may include or be surreounded by a flexible and expandible covering or barrier 300 which may be constructed of, for example, rubber, plastic, corrogated or hinged material, etc. Device 10 may include one or more fins or supports 302 which may hold one more than one shape. For example, fins 302 may have one shape when not under an electric current and may have another shape when under an electric current. In such an embodiment fins 302 may include, for example, nitinol, or other memory shape alloys. Fins 302 may be under the control of, for example, transmitter 20, via wires (not shown). When fins 302 take on one shape, as depicted in FIG. 7B, the appendage of expandible covering or barrier 300 may expand. Dotted line 310 shows the borders of the appendage when the fins 302 are in the configuration of FIG. 7A.

The change in geometry may be triggered by an external signal while the device is being tracked through the body lumen, as discussed above. In an alternate embodiment the change in geometry can take place in response to an internal sensor in the device 10 that senses, for example, time or the surrounding environment. Another example is by pressure measurement that can sense changes in pressure patterns, for example in the GI tract, or a time delay mechanism.

Appendages may include, for example, a bi-morph material, such as polyvinyl. The appendages may change their configuration in accordance with different conditions, as known in the art, such as a temperature or electric voltage gradient. It will be appreciated that the conditions required to cause a configuration change, such as creating a temperature or electric voltage gradient may be externally controlled. Furthermore, appendages may change their configuration when freed from a compressed or restricted form, returning to a previous form.

The packaging surrounding appendages, or appendages, may be implemented by a bimorph material mechanism or shape memory material mechanism, for example polyvinyl or nitinol that may change configuration in accordance with controllable conditions, such as a temperature or electric voltage gradient. The shape memory material, which can be any of the known shape memory alloys or shape memory polymers, may be incorporated, according to an embodiment of the invention, into a covering for appendages or into the appendages so as to enable deflection of the covering or appendages, facilitating a change in shape or configuration of the appendages. The shape memory materials can be bent to various configurations in response to changes in temperature. Thus, different natural or induced in vivo environments having different temperatures can be used to deflect or cause a change in shape of a covering for an appendage or an appendage.

The shape memory material can be caused to change shape using, for example, body heat or possibly heat generated by the device 10. For example, an appendage may include portions including a flexible material such as polyurethane having shape memory capabilities, and may include heat conveying elements, such as one or more wires. Other heat conveying elements may be used. Typically, the heat conveying elements may be connected to a power source at one end and may be embedded in the appendage, and thus may be suitable for effecting a temperature change in the shape memory material. A temperature change may, for example, cause an appendage to deploy or expand, or alternately to contract.

According to one embodiment the appendages are designed such that they are large enough to impart movement to the device without hindering the device movement in the body lumen. For example, an appendage may be the size of a few millimeters up to approximately 25 mm on a device which has a body of about 30 mm. Of course, other suitable sizes for appendages and the device 10 may be used.

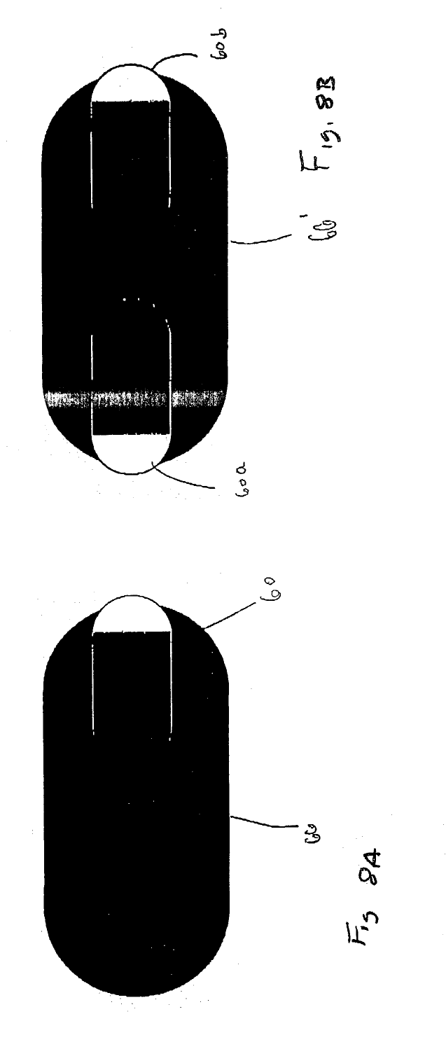

Reference is now made to FIGS. 8A and 8B which are schematic longitudinal cross sectional views of a device 60 with an appendage 66 that substantially surrounds or encompasses the device 60 body. Device 60 is typically a capsule shaped imaging device but may have other shapes and functionality. Appendage 66 is typically capsule shaped, but may have other shapes. The imaging device 60 may include elements as described above.

According to one embodiment, device 60 is ingested while appendage 66 is expanded. Appendage 66 may be collapsed, dissolved or detached after a substantial time delay, for example 100 hours. Other time periods may be used. After such a time delay in the GI tract it may be assumed that the device 60 with the expanded appendage 66 may be obstructed by a stricture. The appendage 66 may be made of a dissolvable material, for example gelatin, that may dissolve after a designated period of time. Once the appendage 66 is eliminated or substantially eliminated, the device 60 may continue through the stricture while, for example, sensing, diagnosing and/or delivering medication to the surrounding area of the stricture.

In an alternate embodiment, device 10 or device 60 includes a tracking and/or movement sensor so that a stricture or other delay may be identified. Suitable tracking devices and methods are described in embodiments of the above mentioned U.S. Pat. No. 5,604,531, or United States patent application publication number US-2002-0173718A1, filed May 20, 2002, entitled “Array System and Method For Locating an In-Vivo Signal Source,” assigned to the assignee of the present invention, and incorporated herein by reference.

Other location and/or orientation detection methods may be used. In one embodiment, the orientation information includes three Euler angles or quaternion parameters; other orientation information may be used. Location and orientation information may be determined by, for example, including two or more transmitting antennas in the above devices, each with a different wavelength, or by detecting the location and orientation using a magnetic method. Methods such as those using ultrasound transceivers or monitors that include, for example, three magnetic coils that receive and transmit positional signals relative to an external constant magnetic field may be used. A GPS or GPS like system may be used; for example a system using transmission from 3 or more stations. If a phase and frequency is used which is high enough (e.g., 300 MHz), a resolution of 1 mm is possible. Other GPS or GPS lice systems may be used.

For example, an array of antennas or sensors may be placed on or close to the abdomen to enable tracking of the capsule. Further, an external command may trigger an alteration in the configuration of appendages, such as the detachment of appendage 66. Once the appendage 66 is removed or reduced, the area of the stricture may be imaged while the device may pass through a strictured area. Appendage 66 or 66′ may be coupled to device 60, 60a and/or 60b, for example, by forming a cast in which devices 60, 60a and 60b are molded.

Detachment may be achieved by, for example, mechanical or electromechanical methods, on command from a controller. Alternately, a glue or dissolveable connector may allow the appendage to detach after a period of time.

In one alternate embodiment, the device may include one or more sensors for sensing the physiological environment around the vicinity of the stricture. In further embodiment, the device may include, for example, a therapeutic device to treat the vicinity of stricture. According to one embodiment (e.g., as described in FIG. 8B) two devices, such as imaging and/or sensing devices 60a and 60b, may be attached or otherwise coupled to an appendage, such as capsule shaped appendage 66′, enabling, inter alia, a wide field of view. According to one embodiment the capsule shaped appendage 66 or 66′ forms a capsule of approximately 26 mm×11 mm. Of course, other suitable shapes and dimensions may be used.

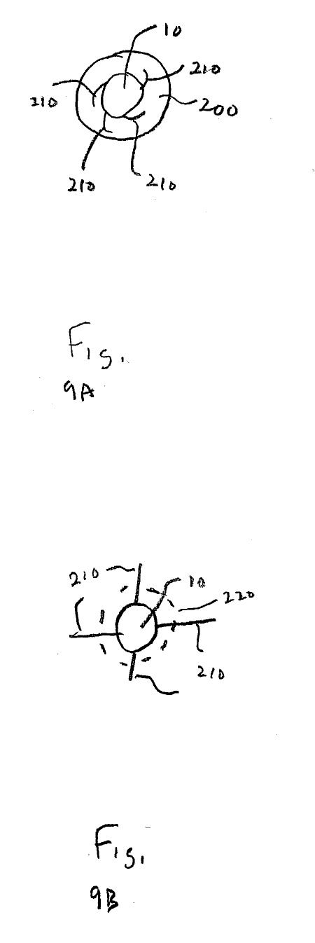

In one embodiment, a device may include a surrounding layer of material which may, for example, dissolve when exposed to a certain pH. Referring to FIG. 9A, there is shown an in-vivo device 10 surrounded by a layer of packaging or material 200. Device 10 includes one or more appendages 210. Appendages 210 typically include a shape memory material (e.g., metal, plastic, etc.) which may be initially bent or folded but, when freed, may extend and take on a different shape. Initially, appendages 210 are folded or bent in a certain configuration, held in place by material 200. When packaging or material 200 dissolves, weakens, or breaks up, appendages 210 are fired and may expand and take on a different shape. FIG. 9B depicts the device 10 after the material 200 has ceased to hold the appendages in place. It can be seen that the appendages 210 expand beyond the border 220 of the device 10 and material 200 prior to the material's dissolving, etc. In one embodiment the material 200 is a material that dissolves or weakens when exposed to low pH, such as that described below. In other embodiments, the material 200 may, for example, dissolve or weaken after a certain amount of time of exposure to liquids, or to a certain temperature (e.g., body temperature).

In one embodiment the material 200 includes an outer coating which may be made of, for example, a Parylene C (typically a dimer of poly p-xylene with a substitution of a single chlorine molecule) coated hydrogel polymer, such as ethyl cellulose acetate and also includes an internal filling which may be made of filler, typically a biodegradable polymer, such as polymer of lactide and golycollide (PLGA). Other materials may be used. The hydrogel polymer creates a matrix that contains the filler and that is strong enough to withstand endo-luminal pressure. The filler absorbs liquid from the body lumen environment which seeps through the hydrogel matrix at a rate which is typically determined by the osmotic gradient between the endo-luminal environment and the inner filling and by properties of the Parylene C coating and of the hydrogel polymer, such as by the extent of the hydrogel polymer cross linking, its concentration, its thickness and so on. The filler swells and after a period of time, starts pressing against the outer coating. The internal pressure rises as more liquid is absorbed. When the pressure reaches a certain, predetermined point the hydrogel matrix and the Parylene C coating rupture and the material 200 is essentially degraded.

According to other embodiments the material 200 may include different hydrogel fillings, which can be induced to go through a change of swelling. For example, a thermo-responsive hydrogel can be stimulated by a change in temperature to go through polymer-polymer and water-polymer interactions, which results in a change in swelling of the hydrogel. Likewise, an acidic or basic hydrogel maybe be induced by a change in pH to swell. According to yet further embodiments the material 200 can be made of materials that are degradable by external methods such as by ultrasound.

According to further embodiments the material 200 may be dissolved/degraded by ultrasound which may be, for example, operated by an external operator.

Encapsulation (e.g., by material 200) may be used to package the appendages by for example a hydrocarbon capsule that dissolves according to specific parameters, such as time, pH, enzymatic activity, temperature, electromagnetic field. For example, material 200 may include a hydrocarbon, such as a caramel or gelatin capsule, which encases the device. The caramel capsule may be dissolved in the liquids present in the stomach, thus releasing the appendages.

The material 200 may be made to dissolve at a specific location along the GI tract. Thus, device 10 may be encased in a hydrocarbon capsule while progressing through certain parts of the GI tract, such as the small intestine, and free of the encapsulation in other parts of the GI tract, such as, the large intestine. While free of the encapsulation device 10 inflated appendages may enable the positioning of the device 10 in the large intestine, for example, away from the walls.

In one embodiment, a control mechanism (e.g., the transmitter 20, as described above, or an external processor) may determine that the device 10 is located in an unfavorable location, such as by a lumen wall, and/or that the device 10 has not moved for a pre-determined period. This may be determined, for example, by comparing consecutive (or non-consecutive) images, for example as described in embodiments described in International Application publication number WO 01/87377, entitled “SYSTEM FOR CONTROLLING IN VIVO CAMERA CAPTURE AND DISPLAY RATE” having the international filing date of May 14, 2001, assigned to the common assignee of the present application and incorporated by reference. In an alternate embodiment the in vivo device may include a tracking mechanism for externally tracking the position and movement of the in vivo device. A command may be sent (e.g. from an internal logic or for example, by using wireless transmission from an external unit) for activating a configuration change. Activation of the configuration change may allow the natural peristaltic movement to advance the in vivo device and more appropriately position the device in the body lumen.

The appendages can also be discarded or otherwise designed to disintegrate so that the device can continue through body lumens with a smaller diameter, for example when passing from the stomach to the small intestine or when passing through strictures in the GI tract or in blood vessels. As discussed above, this may be controlled by an internal controller (e.g., transmitter 20) or an external controller. This can be triggered externally for example when the progression of the device is tracked externally, or internally based on sensing the surrounding environment.

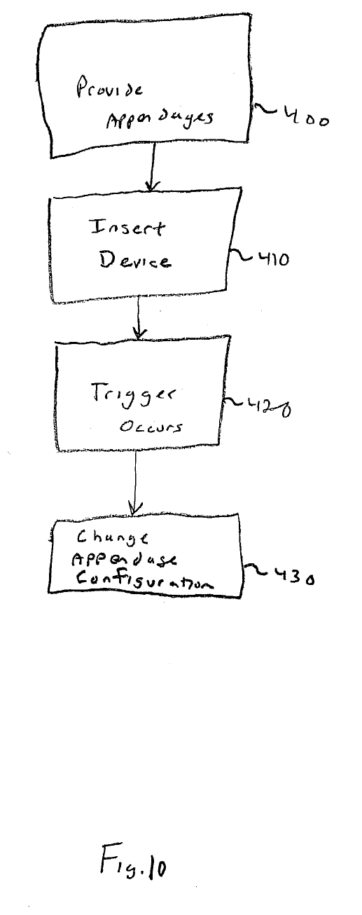

FIG. 10 is a flow chart depicting the steps of a method according to one embodiment of the present invention.

Referring to FIG. 10, in step 400, an in-vivo device is provided with one or more appendages. While typically the appendages may have their configuration changed, they may be static. Whether static or changeable, the appendages may be flexible.

In step 410, the in-vivo device is inserted into a body. For example, it may be ingested.

In step 420, a trigger may occur. For example, a time limit may pass, or an environmental condition may be sensed (e.g., a change in pH).

In step 430, the appendages may have their configuration changed. For example, the appendages may be expanded, shrunk, dissolved, etc.

In other embodiments, other steps or series of steps may be used. For example, the appendages need not have their configuration changed or a trigger need not occur. Furthermore, the configuration may be changed in a passive manner—e.g., by the appendages or a cover dissolving or responding to in-vivo conditions.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. An in-vivo device comprising:
a housing;
a sensor contained within the housing; and
an appendage coupled to and extending from the housing, wherein the size or configuration of said appendage is changeable.

2. The device according to claim 1, wherein the housing is substantially rigid and the appendage is flexible.

3. The device according to claim 1, wherein the appendage is expandable or extendible.

4. The device according to claim 1, wherein the appendage is capable of detaching, collapsing, or disintegrating.

5. The device according to claim 1 wherein the appendage is selected from a group comprising one or more of the following: wing shaped, disc shaped, cone shaped, capsule shaped or any combination thereof.

6. The device according to claim 1 wherein the appendage is substantially symmetric relative to a longitudinal axis of the device.

7. The device according to claim 1 wherein the appendage is to position the device housing at an orientation substantially parallel to a body lumen wall.

8. The device according to claim 1 wherein the appendage is to position the device housing at an angle relative to a body lumen wall.

9. The device according to claim 1 wherein the appendage includes one or more of the following: rubber, silicon, hydrocarbon.

10. The device according to claim 1 wherein the appendage includes a bimorph material.

11. The device according to claim 1 comprising a packaging surrounding the appendage.

12. The device according to claim 11, wherein the packaging is dissolvable.

13. The device according to claim 1 wherein the appendage may be controlled based on sensing one or more of the following: pressure, temperature, pH, and enzymatic activity.

14. The device according to claim 1 comprising a transmitter.

15. The device according to claim 1 comprising a receiver.

16. The device according to claim 1 wherein the sensor includes an image sensor.

17. The device according to claim 1 comprising a location tracking device.

18. The device according to claim 1, comprising a set of substances which, when combined, produce gas.

19. The device according to claim 1, wherein the appendage includes at least a flexible and expandable covering.

20. A system for in vivo sensing, the system comprising:
a device body;
a sensor disposed within the device body;
at least one appendage coupled to the device body; and
a transmitter.

21. An in-vivo sensor comprising:
a body;
an imager disposed within the shell; and
an extendible appendage.

22. An in vivo device comprising:
a housing means for housing an in vivo sensor; and
an appendage means for positioning the in vivo device with respect to a body lumen.

23. A method for positioning an ill-vivo device within a body lumen, the method comprising:
providing an in-vivo device with an appendage; and
inserting the device in vivo.

24. The method of claim 23 comprising extending the appendage in vivo.

25. The method of claim 23 comprising:
sensing an in-vivo condition; and
on the detection of the condition, causing the extending of the appendage.

26. The method of claim 23 comprising:
sensing an in-vivo condition; and
on the detection of the condition, causing the collapse of the appendage.

27. The method of claim 23, wherein the device includes substantially rigid housing and the appendage is flexible.

28. The method of claim 0.23 comprising positioning the device at an orientation substantially parallel to a body lumen wall.

29. The method of claim 23 comprising dissolving or removing a packaging surrounding the appendage.

30. The method according to claim 23 comprising capturing images.

31. The method according to claim 23 comprising transmitting data.

32. The method according to claim 23 comprising transmitting position information.

33. A method for positioning an in vivo device within a body lumen, the method comprising the steps of:
inserting the device in vivo, said device comprising an appendage; and
collapsing the appendage in vivo.

34. A method for in-vivo imaging, the method comprising the step of:
on the occurrence of an in vivo environmental condition, altering the configuration of an appendage attached to an in-vivo imaging device.

35. A method for in-vivo sensing, the method comprising:
inserting into a body an in-vivo device, the device including a sensor and including an appendage; and
altering the configuration of the appendage.
